(19) United States
(12) Patent Application Publication
(10) Pub. No.: US 2012/0035544 A1
(43) Pub. Date: Feb. 9, 2012

(54) MEDICAL DEVICES WITH PLASMA-TREATED SURFACE AND METHODS

(75) Inventors: Kimberly A. Chaffin, Woodbury, MN (US); Geetha Sethumadhavan, Plymouth, MN (US)

(73) Assignee: MEDTRONIC, INC., Minneapolis, MN (US)

(21) Appl. No.: 12/850,278

(22) Filed: Aug. 4, 2010

(51) Int. Cl. A61M 5/142; A61N 1/362; B32B 37/00; A61M 5/315; A61M 5/00; A61N 1/36; A61N 1/39

(52) U.S. Cl. 604/151; 607/2; 607/36; 607/5; 604/218; 604/246; 156/272.2

(57) ABSTRACT

A medical device (e.g., an implantable medical device) including a sealing apparatus (sealing element, e.g., a grommet for securing a lead to the device) that includes an element (e.g., a body) having a plasma-treated surface and methods (e.g., methods of making the device).

### BACKGROUND

Silicone rubber is used widely in the medical industry for the manufacture of various components for implantable and non implantable applications. The widespread use of silicone rubber is due to excellent biocompatibity and biostability properties, and the fact that it can be processed by molding, extrusion, etc.

Silicone rubber (PDMS: Polydimethylsiloxane) has low surface energy. Therefore the adhesion of silicone rubber to other substrates and to itself is very weak when placed in contact with each other for short periods of time in absence of pressure. However, in silicone rubbers, a bulk healing phenomenon called silicone blocking or self adhesion can occur over time. Blocking is accelerated with stress. Siloxane bonds (Si—O) can interchange to allow for stress relaxation in the silicone. This bond interchange (or healing) results in zero stress level locally in the polymer. Molecularly, siloxane bond (Si—O) rearrangement takes place, moving the Si—O bond from one chain to another. There is no change in molecular weight, but only in bonding structure and stress level. The following shows the bond exchange that happens that causes stress relaxation and blocking in silicone rubber.

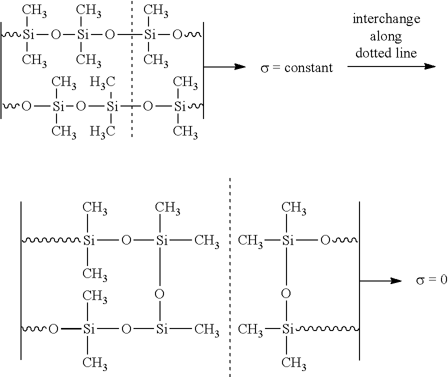

As an example, silicone blocking can occur at a small cut/slit when stress is present, resulting in a healing of the cut/slit over time. Although silicone rubber is in a cured, crosslinked state, when low levels of stress are present the siloxane bonds are still dynamic. As a result of blocking, the components made with silicone rubber could be rendered inoperable because the required slit heals over time. This can occur, for example, in the pump tubes of an implantable drug pump. During delivery of a drug, the pump tube is compressed by roller arms which rotate to move the drug through the pump tube. During manufacture of drug pumps, after the pump tube is installed inside the drug pump casing, if the device is idle with the pump head not turning, the pump head roller compresses the pump tube. This compression of the pump tube causes the inside walls of the pump tube to stick together (i.e., set or block) over time.

In another example, stimulation devices for various implantable applications have a molded silicone rubber component called a grommet, which is comprised of two symmetrical halves. After connecting the leads or extensions in the devices, the physicians use a torque wrench inserted between the two halves of the grommet for accessing and tightening the setscrews to hold the leads or extensions in contact with electrical contacts in the devices. A common problem that is encountered in the devices is the healing or blocking of the slit in the grommet during storage prior to implantation. The healing or blocking of the grommet slits leads to the setscrews being inaccessible or the silicone rubber tearing when the torque wrench is inserted in the grommet.

Many devices also use valves, which are molded using silicone rubber, and include a slit placed in the valve prior to post cure. This slit can heal during post cure operations due to silicone blocking. Also, silicone rubber components for use in various medical devices are molded in batches of large quantities and are often packaged in bags for shipping. There have been issues noted with molded silicone components (for example, pieces of tubing) sticking to each other over time.

This problem can also occur in components in which silicone is placed against silicone, silicone is placed against other materials such as glass, metal, or other organic polymers. This problem can also occur with other elastomeric polymers such as EPDM (ethylene propylene diene monomer), butyl rubber, or fluorine elastomers, which are placed against each other or other materials.

Therefore, it is important to understand the causes for such blocking and come up with solutions to reduce such blocking.

### SUMMARY

The present invention provides a medical device and methods. A medical device (e.g., an implantable medical device) of the present invention includes a sealing apparatus (sealing element, e.g., a grommet for securing a lead to the device) that includes an element (e.g., a body) having a plasma-treated surface. Preferably, the plasma-treatment serves to immobilize mobile species (e.g., oligomers, surfactant) at the plasma-treated surface. Significantly, this can serve to reduce the blocking between the plasma-treated surface and a second surface, which may or may not be plasma-treated. This blocking between the two surfaces is the undesired adhesion/self-healing of two elements at the contact surface (i.e., the interface).

In one embodiment, the present invention provides a medical device (preferably, an implantable medical device) including a sealing apparatus that includes a first element and a second element, wherein the first element includes a first surface and the second element includes a second surface, wherein the first surface of the first element faces and is in physical contact with the second surface of the second element, and wherein the first element includes an organic polymer and has a bulk durometer value on the Shore A scale (e.g., a value of at least 30 A), and further wherein the first surface is a plasma-treated surface. In certain embodiments, the second element includes an organic polymer and has a bulk durometer value on the Shore A scale (e.g., a value of at least 30 A). In certain embodiments, the first element and the second element comprise portions of a single, integral body. In certain embodiments, each of the first and second surfaces is a plasma-treated surface.

In another embodiment, there is provided a medical device including a sealing element, wherein the sealing element includes two surfaces forming an interface under a compressive stress, wherein at least one surface at the interface comprises an organic polymeric material having a bulk durometer value on the Shore A scale (e.g., a value of at least 30 A) and is plasma-treated. In certain embodiments, the two surfaces forming an interface are polymeric surfaces (e.g., silicone surfaces).

The present invention also provides methods. In one embodiment, there is provided a method of making a medical device that includes a sealing apparatus, the method includes: providing a first element including an organic polymer having a bulk durometer value on the Shore A scale (e.g., a value of at least 30 A), wherein the first element has a first surface; providing a second element having a bulk durometer value on the Shore A scale (e.g., of at least 30 A), wherein the second element has a second surface; treating the first surface of the first polymeric element with a plasma to form a plasma-treated surface; and contacting the plasma-treated surface of the first polymeric element with the second surface of the second element to form a sealing apparatus. Preferably, treating the first surface with a plasma creates an oxygen-rich surface. Typically, treating the surface with a plasma is carried out for a time sufficient to prevent blocking.

The terms “comprises” and variations thereof do not have a limiting meaning where these terms appear in the description and claims.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably.

As used herein, the term “or” is generally employed in its sense including “and/or” unless the content clearly dictates otherwise.

The term “and/or” means one or all of the listed elements or a combination of any two or more of the listed elements (e.g., preventing and/or treating an affliction means preventing, treating, or both treating and preventing further afflictions).

Also herein, all numbers are assumed to be modified by the term “about” and preferably by the term “exactly.” Notwithstanding that the numerical ranges and parameters setting forth the broad scope of the invention are approximations, the numerical values set forth in the specific examples are reported as precisely as possible. All numerical value, however, inherently contain certain errors necessarily resulting from the standard deviation found in their respective testing measurements. That is, as used herein in connection with a measured quantity, the term “about” refers to that variation in the measured quantity as would be expected by the skilled artisan making the measurement and exercising a level of care commensurate with the objective of the measurement and the precision of the measuring equipment used.

Also herein, the recitations of numerical ranges by endpoints include all numbers subsumed within that range (e.g., 1 to 5 includes 1, 1.5, 2, 2.75, 3, 3.80, 4, 5, etc.).

As used herein, the term “room temperature” refers to a temperature of about 20° C. to about 25° C. or about 22° C. to about 25° C.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1: A typical grommet used in an implantable medical device.

FIG. 2: A typical valve having a valve slit used in an implantable medical device.

FIG. 3: A graph showing blocking over time for materials having different durometer values.

FIG. 4: A graph showing the effect of Supercritical CO₂ cleaning on blocking.

FIG. 5: A graph showing a comparison of blocking in a Control versus a Supercritical CO₂ cleaned material.

FIG. 6: A graph showing a comparison of blocking in Control versus a Supercritical CO₂ cleaned material.

FIG. 7: A graph showing the effect of plasma treatment on blocking.

FIG. 8: A graph showing a comparison of O/C ratio and contact angle.

FIG. 9: A representation of low molecular weight species in silicone rubber (oxygen in air harnessing oligomers at surface).

FIG. 10: A representation of a mechanism for hydrophobic recovery in plasma-treated PDMS.

FIG. 11: A representation of the formation of oxygen rich layer on plasma-treated silicone rubber.

FIG. 12: A representation of blocking in Liquid Silicone Rubber (LSR, Control).

FIG. 13: A representation of blocking in plasma-treated LSR.

### DETAILED DESCRIPTION OF ILLUSTRATIVE EMBODIMENTS

The present invention provides a medical device and methods for reducing or preventing undesirable blocking of surfaces. In this context, “blocking” is the undersized adhesion/self-healing of the two elements at a contact surface (i.e., the interface) such that the elements do not pull apart with 2 pounds/inch without tearing one or both elements. This occurs frequently, for example, with a silicone-silicone surface, although it can also occur with a silicone-glass surface, a silicone-metal surface, a silicone-polymer surface wherein the polymer is an organic polymer (typically, an elastomer) other than silicone (e.g., ethylene propylene diene monomer, butyl rubber, fluorine elastomers or combinations thereof). This can also occur with elastomeric polymers such as ethylene propylene diene monomer, butyl rubber, fluorine elastomers, and combinations thereof at interfaces with similar elastomeric materials or materials such as glass and metal. Significantly, the present invention provides a mechanism to overcome this problem of blocking by plasma-treating one or both surfaces at the interface.

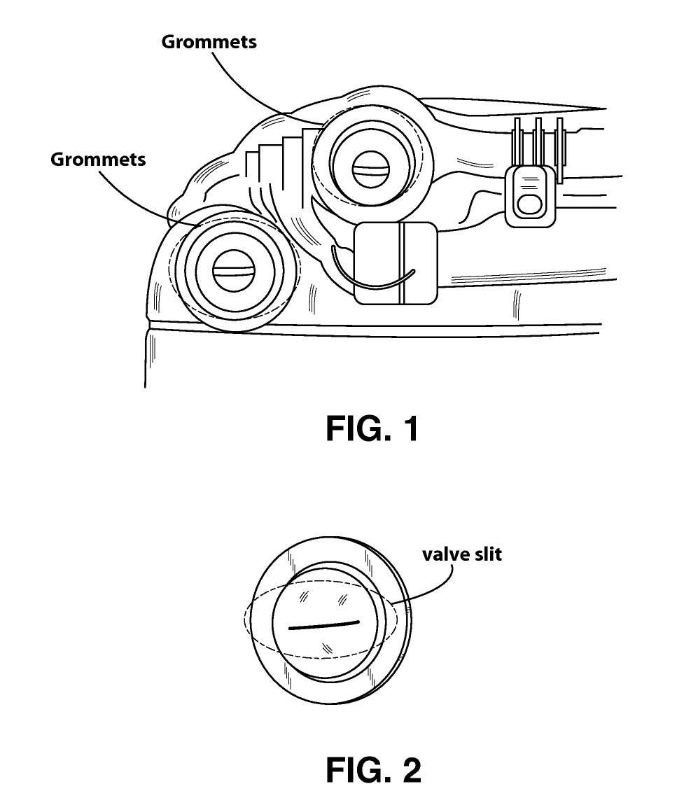

The present invention thus provides advantageous medical devices. The device is preferably an implantable medical device. Preferred implantable medical devices are “can” devices such as stimulators, pacemakers, defibrillators, drug pumps, and implantable pulse generators. Such devices include a sealing apparatus (sealing element), such as a grommet for securing a lead to the device (as shown in FIG. 1), a plunger in a syringe, a heart valve, or a valve slit (as shown in FIG. 2), for example, or other such fastener that forms a seal (e.g., a fluid-tight connection), typically because of its pressure geometry and shape.

Typically, such components of a medical device include a first element with an organic polymeric surface that forms an interface with a second element with a second surface, wherein such interface is under a compressive stress. In such a situation, without the present invention, “blocking” occurs at the interface. These elements that form the interface can be of the same material or of different materials, they can be part of one body or they can be separate bodies joined to form the interface. Typically, both elements have surfaces that are made of organic polymeric materials (e.g., silicone surfaces), but blocking can occur between an organic polymer and another (e.g., glass, metal) material.

Thus, the present invention provides a sealing element (i.e., sealing apparatus), which is typically used in a medical device, particularly an implantable medical device, wherein the sealing element includes two surfaces (e.g., a first surface of a first body or first element and a second surface of a second body or second element). At least one of these surfaces, and preferably both of these surfaces, is made of an organic polymeric material having a bulk durometer value on the Shore A scale (e.g., a value of at least 30 A). These surfaces form an interface (e.g., the first surface of the first element faces and is in physical contact with the second surface of the second element) under a compressive stress, wherein at least one surface at the interface is a plasma-treated surface. If only one surface is made of an organic polymeric material and the other is of another material (e.g., glass, metal), the organic polymeric material is typically that which is plasma-treated.

In a preferred embodiment, the present invention provides a medical device (preferably, an implantable medical device) including a sealing apparatus that includes a first element and a second element, wherein the first element includes a first surface and the second element includes a second surface, wherein the first surface of the first element faces and is in physical contact with the second surface of the second element, and wherein the first element includes an organic polymer and has a bulk durometer value on the Shore A scale (e.g., a value of at least 30 A), and further wherein the first surface is a plasma-treated surface. Preferably, the second element includes an organic polymer and has a bulk durometer value on the Shore A scale (e.g., a value of at least 30 A),

In certain embodiments of the present invention, the material at the interface has a bulk durometer value on the Shore A scale. In certain preferred embodiments, this value is at least 30 A. In certain preferred embodiments, this value is no greater than 70 A. The Shore A durometer scale is a well-known hardness test measured according to the test procedure ASTM D2240. The A scale is for relatively soft materials, but do not include really soft materials such as chewing gum or pressure sensitive adhesives. For example, materials that have Shore A values include a rubber band (e.g., having a Shore A value of 25), a pencil eraser (e.g., having a Shore A value of 40), a car tire tread (e.g., having a Shore A value of 70). Materials used in typical sealing elements have Shore A values, for example, of 30, 50, or 70.

In certain embodiments, the first element (or first body) and the second element (or second body) comprise portions of a single, integral body. In certain embodiments, each of the first and second surfaces is a plasma-treated surface. Either or both elements (or surfaces) include a mobile species that is immobilized at the plasma-treated surface. Such species can be inherent in the polymer (e.g., an oligomeric species within the polymer) or it can be an additive (e.g., a surfactant added to the polymer). For example, silicone includes oligomers having a molecular weight less than the entanglement molecular weight of the silicone, and the interface between two silicone surfaces comprises a region of a higher concentration of the oligomers relative to the remainder of the silicone.

This immobilization occurs as a result of surface energetics of the plasma used to treat the surface(s). The interface containing the harnessed mobile species does not allow for the creation of a bond between the surfaces that has any mechanical consequences.

In certain embodiments, the sealing apparatus is a grommet for securing a lead to the device, as shown in FIG. 1. As is well-known to one of skill in the art, a grommet is used to electrically insulate a connection between a lead and the body of a patient. Typically, in a grommet the interface is formed of two silicone surfaces.

Alternatively, the sealing apparatus can be a plunger in a syringe. Typically, in a syringe, the plunger is made of an organic polymer and body of the syringe against which the plunger forms a sealing interface is made of a metal, glass, or another organic polymer.

Alternatively, the sealing apparatus can be a valve wherein the first and second surfaces form a valve slit, as shown in FIG. 2. Typically, in a valve the interface is formed of two surfaces made of the same or different organic polymeric materials (e.g., silicone).

Thus, in certain embodiments of a medical device of the present invention, a first element includes silicone, ethylene propylene diene monomer, butyl rubber, fluorine elastomers, and combinations thereof.

In certain embodiments of a medical device of the present invention, a second element includes a metal, glass, or an organic polymer. In certain embodiments of a medical device of the present invention, a second element includes an organic polymer. In certain embodiments of a medical device of the present invention, the first and second elements comprise silicone and the first and second surfaces form an interface under a compressive stress. For example, the first and second surfaces form an interface and display a lower peel strength 48 hours after formation of the interface than a control, wherein the control includes an interface formed of the same first and second elements (having the same first and second surfaces) without the plasma-treated surface. This can also be demonstrated for grommets by performing a Grommet Punchout Test, as described herein. A high level of blocking causes a wrench to punch out a plug of material (e.g., silicone) when the bottom of the grommet contacts the set screw. This is a punch and die effect and is undesirable.

The present invention also provides methods. In one embodiment, there is provided a method of making a medical device that includes a sealing apparatus, the method includes: providing a first element including an organic polymer having a bulk durometer value on the A scale (e.g., a bulk durometer value of at least 30 A), wherein the first element has a first surface; providing a second element, wherein the second element has a second surface; treating the first surface of the first organic polymeric element with a plasma to form a plasma-treated surface; and contacting the plasma-treated surface of the first polymeric element with the second surface of the second element to form a sealing apparatus. In certain preferred methods, the second element includes an organic polymer having a bulk durometer value on the A scale. In certain preferred methods, the first element includes an organic polymer and has a bulk durometer value of at least 30 A, and wherein the second element includes an organic polymer and has a bulk durometer value of at least 30 A.

Typically and preferably, treating silicone surface(s) with a plasma creates an oxygen-rich surface, which pins low molecular weight silicone oligomers at the interface. That is, a plasma-treated silicone surface has a higher O/C ratio than exists within the bulk of the silicone. This oxygen-rich surface creates an energetically favorable position for low molecular weight species, for example, to be attracted to the surface, and hence, an interface with another surface, particularly another plasma-treated surface. Thus, at an interface between two materials wherein at least one surface is plasma-treated, the ratio of O/C is higher than in the bulk of the material (particularly immediately after plasma treatment), as is the concentration of low molecular weight species (particularly over time). This is believed to help prevent or reduce the amount of blocking.

Other materials may require different surface energetics to immobilize mobile species at their interfaces. Such different surface energetics might be derived from other surface chemistries as would be readily determined by one of skill in the art based on the teachings herein.

The methods used to create the medical devices and sealing apparatuses herein can include a variety of techniques. One way to deposit oxygen (e.g., chemically attach oxygen) to the surface of silicone is through the use of a plasma, such as radio frequency induced plasma. Other techniques can include, for example, chemical etching, chemical washing, For example, in treating a silicone surface, a plasma can be used that is generated at a frequency of about 13.56 MHz and at 150 mTorr, although pressures of higher or lower values can also be used to generate a plasma effective for the present application. The plasma treatment can take place in the presence of a gas selected from the group consisting of hydrogen, nitrogen, helium, argon, neon and mixtures thereof.

Typically, treating the surface with a plasma is carried out for a time sufficient to prevent blocking. Typically, sufficient time is used to create surfaces that form an interface and display a lower peel strength 48 hours after formation of the interface than a control, wherein the control includes an interface formed of the same first and second elements without the plasma-treated surface.

### EXAMPLES

Objects and advantages of this invention are further illustrated by the following examples, but the particular materials and amounts thereof recited in these examples, as well as other conditions and details, should not be construed to unduly limit this invention.

The purpose of the present research was to study the rate of blocking in silicone rubber. The first objective was to study the effect of durometer on rate and extent of blocking in silicone rubber. The second objective was to study the effect of argon plasma surface treatment on the rate of blocking. The third objective was to study the effect of supercritical CO₂ cleaning on the rate of blocking.

The following experimental results showed that with increase in durometer of silicone rubber, the rate of blocking decreased. It is believed that this is due to reduced molecular mobility because of the higher crosslink density and filler amount in the higher durometer silicone rubber. Surface treatment with argon plasma prevented blocking in silicone rubber. Plasma treatment created an oxygen rich SiO₂ layer on the surface and caused migration of low molecular species to the surface over time due to high surface energetics of the oxidized surface. The near surface SiO₂ layer remained present during the blocking experiment, keeping the energetics favorable for the silicone oligomers to remain at the interface. The consequence was a weak boundary layer that prevented blocking. The presence of SiO₂ layer and hydrophobic recovery was confirmed with ESCA and contact angle measurements. Removal of low molecular weight species in silicone rubber using supercritical CO₂ cleaning increased rate of blocking. Removal of low molecular weight species from the silicone rubber prevented the formation of a weak boundary layer on the surface that reduces adhesion strength at the interface.

### Blocking Experiment Setup

### Materials Used for Blocking Experiment

The materials used for the blocking experiments were Silastic BioMedical Grade Liquid Silicone Rubber (LSR) from Dow Corning Corporation. The specific LSR materials that were used were 7-6830 (30 A), 7-4870 (70 A), and 7-4850 (50 A). The properties of the three materials generated and reported by the vendor are listed below in Table 1.

TABLE 1
Properties of LSR materials (from Dow Corning specification sheet) used
for blocking experiments

| | 7-6830 | 7-4850 | 7-4870 |
|---|---|---|---|
| Durometer (Shore A) | 30 | 53 | 66 |
| Elongation | 790% | 630% | 420% |
| Tear Strength (pounds per inch: ppi) | 140 | 260 | 270 |
| Tensile Strength (pounds per square inch: | 1280 | 1470 | 1380 |
| psi) | | | |

The LSR consists of Part A and Part B. Part A has the platinum catalyst for the cure and other proprietary components. Part B has the silicone polymer crosslinker and inhibitor. The polymer is crosslinked through addition cure at elevated temperature. Fumed silica is used as the filler in the polymer.

### Material Characterization

### Tear Strength for LSRs

The load extension curves for the three liquid silicone rubber (LSR) materials used were generated using MTS tester. ASTM method D624 was used for the tear strength testing and utilized Die type B. Testing was performed using a speed of 20 inches/minute.

### Material Configuration

The LSR materials used for the study were molded as slabs. The slab dimensions were 6 inches long by 6 inches wide by 0.125 inch thick. The slab for the blocking experiment had pieces of stainless steel mesh embedded in the middle and the mesh extended past the molded rubber part by an inch. This mesh gave the slab rigidity and prevented stretching of the silicone material when performing the self adhesion strength testing. The portion of the mesh that extended past the molded rubber allowed for clamping during the adhesion strength testing. The slabs used for material characterization were molded without the stainless steel mesh.

The mesh consisted of a rectangular stainless steel sheet (8-inch long by 2-inch wide; 0.0045-inch wire thickness; cleaned in citric acid to remove oils). The mesh (part number: R8.00X2.00S100X.0045) was purchased from TWP Inc (Berkeley, Calif.). The slabs were molded at Dow Corning using LSR material. The cure parameters that were used were 302° F. for 10 minutes. Per the vendor, the slabs were not required to be post cured after molding. Each slab was cut into twelve (12) bars that were 3 inches long×1 inch wide×0.125 inch thick. Each piece had an inch of protruding mesh on one side. Two bars of molded silicone were used for each experiment.

### Experiment Method

The surfaces of the molded silicone bars were wiped clean using 70/30 isopropyl alcohol/water mixture and dried using an air gun to remove any surface contaminants. The two bars were placed in contact with each other and placed in the mold.

A small piece of Teflon was placed between the silicone bars at one end to separate the interface and prevent self adhesion near the tab used for gripping in the experiment. A two plate Aluminum mold was used for the experiment. Each mold plate had cavities for the bars of silicone to be seated. The cavities were 0.0625-inch deep. The two silicone bars were placed in the mold and the mold placed in a Wabash hydraulic heat press (Manufacturer: Wabash MPI, Wabash, Ind.). The press is capable of operating up to 10,000 psi. All the adhesion experiments were performed using the heat press located in a Class 10,000 cleanroom. The humidity was controlled between 30% and 40%. The temperature was also controlled between 60° F. and 80° F.

The platens of the heat press were heated to 400° F. for all the testing conducted. The pressure applied on the mold was 83 psi. The mold with the two silicone samples was placed in the middle of the platen so that any variability due to uneven pressure was avoided throughout the experimentation. The mold with the silicone samples was left in the heat press at high temperature/pressure for different periods of time. At various time points, the mold was removed from the press. The samples were removed from the mold and the Teflon piece removed. The samples were allowed to cool in air. The samples were trimmed to 0.5-inch width (0.25-inch on both sides were trimmed off) to eliminate the edge effect that occurred in some of the samples.

### Adhesion Strength Measurement

Adhesion strength measurements were performed in a MTS tester. The method used for the adhesion measurement was T-peel method. The silicone bars (one on the top grip and the second bar on the bottom grip) were mounted in the MTS and the samples were peeled at a speed of 0.1 inch/minute. The load-extension data was obtained for each run. The average of the load over the flat portion of the curve was calculated. Since the width of the sample was fixed at 0.5 inch, the average peel strength was calculated as Average Load/Peel width (0.5). This value was reported as the Peel Strength in units of pounds/inch.

### Supercritical CO₂ Cleaning

For the purpose of the study, LSR with medium rates of blocking (7-4850 LSR) was chosen.

### Characterization of Control and Supercritical CO₂ Cleaned 7-4850 LSR

Measurement of catalyst amount in 7-4850 LSR samples. The amount of platinum in control 7-4850 material and supercritical CO₂ cleaned 7-4850 LSR material was measured using Inductively Coupled Plasma (ICP) analysis. The material was weighed into ceramic crucibles and ashed in a furnace at 550° C. for 2 hours. The ashed sample was transferred to polypropylene block digestion tubes and digested in a digestion block using hydrofluoric acid and aqua regia. The samples were analyzed using a Perkin-Elmer Inductively Coupled Plasma-Optical Emission Spectroscopy (ICP-OES) Optima 5300DV. The instrument was calibrated with a certified National Institute of Standards (NIST) traceable standard using a blank and two levels of Pt to establish a calibration curve. The samples were analyzed and quantified against the calibration curve.

Soxhlet Extraction of 7-4850 LSR. Soxhlet extraction was carried out on the control 7-4850 LSR. The solvent used was heptane. The experimental setup included a variable autotransformer, stir plate, heating mantle, round bottom flask for the heptane, soxhlet extractor and condenser. The extraction was performed for 29 hours using heptane. The cycle time for the extraction was 5 minutes.

GPC Analysis of Extract from Control 7-4850 LSR. The extract from soxhlet extraction of the Control 7-4850 LSR was analyzed using the Gel Permeation Chromatography (GPC) technique. The solvent (heptane) was removed from the extract using a rotary vacuum equipment. The molecular weight distribution of the extract was determined using the GPC refractive index (RI) detection.

Experimental Setup
Equipment: Waters GPCV2000 with Millenium software
Chemical used: Toluene (0.7 ml/minute)
Polystyrene standards
Columns: Three Waters Ultrastyragel: HR 0.5, HR 3, and 10̂4 A
Column temperature: 50° C.
Sample ID: R200902040 (Control)

Plasma Treatment. The plasma treatment of 7-4850 LSR was carried out in a bell jar reactor. Radio frequency (RF, 13.56 MHz) power was delivered through an arrangement of one powered and two grounded planar electrodes. Applied and reflected power was balanced using a matching network. Pressure was measured with a Baratron sensor placed between the reactor and the vacuum pump. The pressure was controlled with a throttle valve placed between the pressure sensor and the pump. Argon was metered into the chamber with mass flow controllers.

Silicone test samples were placed on the powered electrode. The treatment chamber was pumped down to a base pressure of approximately 10 mTorr. Reactant gases were metered into the system with a flow rate of 5 std cubic cm/minute to an operating pressure of 150 milliTorr. The reactor was allowed to equilibrate for 10 minutes. 80 W Watts of RF power was applied to the system for 5 minutes. With the RF power off, the chamber was then brought up to atmospheric pressure.

### Characterization of Control and Plasma-Treated 7-4850 LSR

Contact Angle Measurements. Contact angle measurements were performed using Rame Hart Goniometer at different time points after the plasma treatment of 7-4850 LSR and compared to the control sample. The sessile drop method was used to measure the static contact angle. Contact angle measurements were made using water in air. The contact angle measurements are the mean of the left and right contact angle, calculated by the Drop image software. The setup used for the contact angle measurements included the Rame Hart Goniometer with the Auto Pipetting system and the Drop Image Software. The volume of the water drop was set at 4 microlitre.

Surface Analysis using Electron spectroscopy for chemical analysis (ESCA). Electron spectroscopy for chemical analysis (ESCA) also known as x-ray photoelectron spectroscopy (XPS) was used for the surface analysis of the silicone rubber samples. The samples that were used were control 7-4850 (50 A LSR), plasma-treated 7-4850 (50 A LSR), extracted 7-4850 (50 A LSR), and plasma-treated extracted 7-4850 (50 A LSR). The extracted samples were extracted using soxhlet extraction method using heptane as the solvent.

A survey spectrum to, determine all elements present (except H) was first acquired from each analysis area. The spectra were used to obtain quantitative surface composition by integrating the areas under the photoelectron peaks and applying empirical sensitivity factors. High energy resolution ESCA of the Si2p and C1s peaks was used to determine the Si2p binding energy. The depth of analysis of this technique was on the order of 10 nm with take off angle of 45 degrees.

### Blocking Experiment Results

### Tear Strength for LSR Materials

A summary of the key properties of the three liquid silicone rubber (LSR) materials used for the study are listed in Table 2. Tear strength was calculated as Load at Tear/width of the sample. Tear strength results show that as durometer increases, tear strength increases.

TABLE 2
Summary from LSR Tear Strength Testing

| | | 7-6830 | 7-4850 | 7-4870 |
|---|---|---|---|---|
| Width | in | 0.074 | 0.078 | 0.078 |
| TearStrength | lbf/in | 239.62 | 276.43 | 290.05 |
| PeakLoad | lbf | 17.61 | 21.56 | 22.48 |
| LoadAtTear | lbf | 17.61 | 21.56 | 22.48 |

### Effect of Durometer of Liquid Silicone Rubber on Blocking

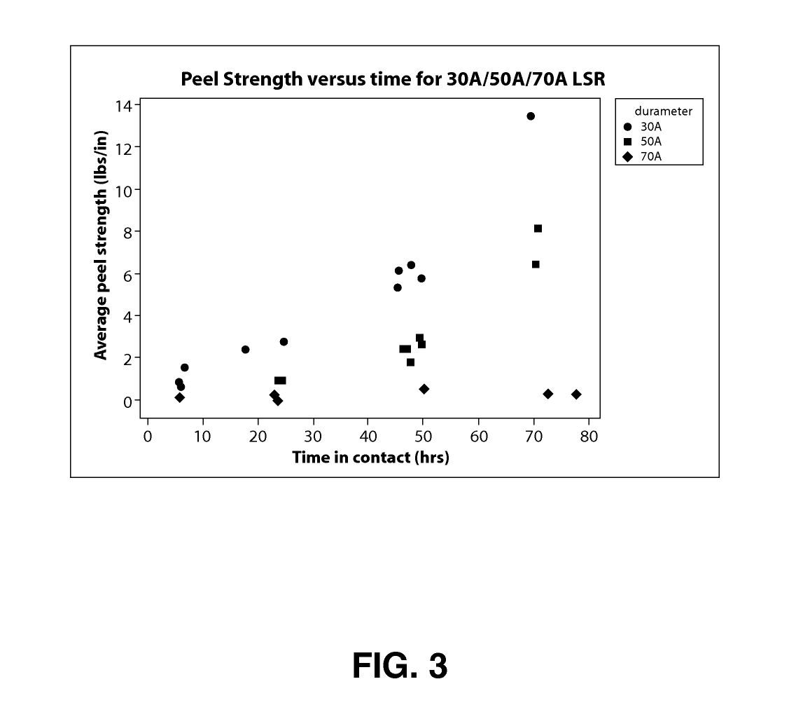

FIG. 3 shows the blocking data for different durometers of LSR over time. In all cases the failure mode during the peel testing was the clean separation of the interface. That is, the silicone slabs failed at the interface and not cohesively in the bulk of silicone rubber. The graph shows that self adhesion or blocking increases with time for 30 A and 50 A LSR materials. The 30 A LSR exhibited more blocking than 70 A or 50 A LSR. The 70 A LSR material showed much less blocking.

The 30 A material has lower crosslink density than 70 A or 50 A material. Also the filler amount is higher in 70 A. The results indicate that blocking occurs at a faster rate in material that has lower crosslinked density and filler amount.

ANOVA (Analysis of Variance) was performed using Minitab 15 on the peel strength data for the three (3) different durometers—30 A, 50 A and 70 A at time point 48 hours. The ANOVA results (not shown) indicated that there is a statistically significant difference in peel strength for the different durometers, since the p value is less than 0.5.

### Effect of Supercritical CO₂ Cleaning on Blocking

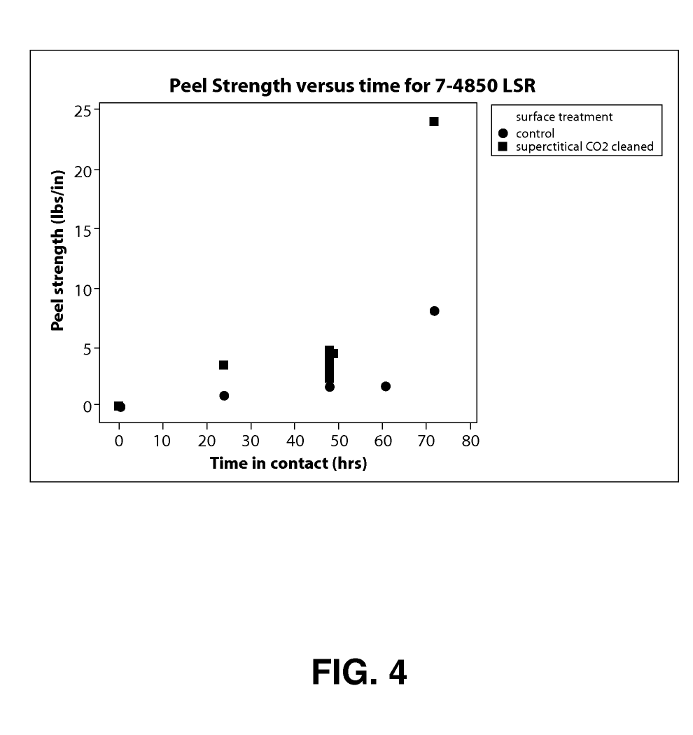

FIG. 4 shows that self adhesion or blocking increases with time for both supercritical CO₂ cleaned 7-4850 LSR and control 7-4850 LSR. Supercritical CO₂ cleaning significantly increased the blocking rate in the 7-4850 LSR at all the different time points studied in the experiment. In all cases (with the exception of the supercritical CO₂ cleaned sample at 72 hours) the failure mode during the peel testing was the clean separation of the interface. That is, the silicone slabs failed at the interface and not cohesively in the bulk of silicone rubber. In the case of the supercritical CO₂ cleaned sample at 72 hours, the failure mode was cohesive failure in the silicone rubber.

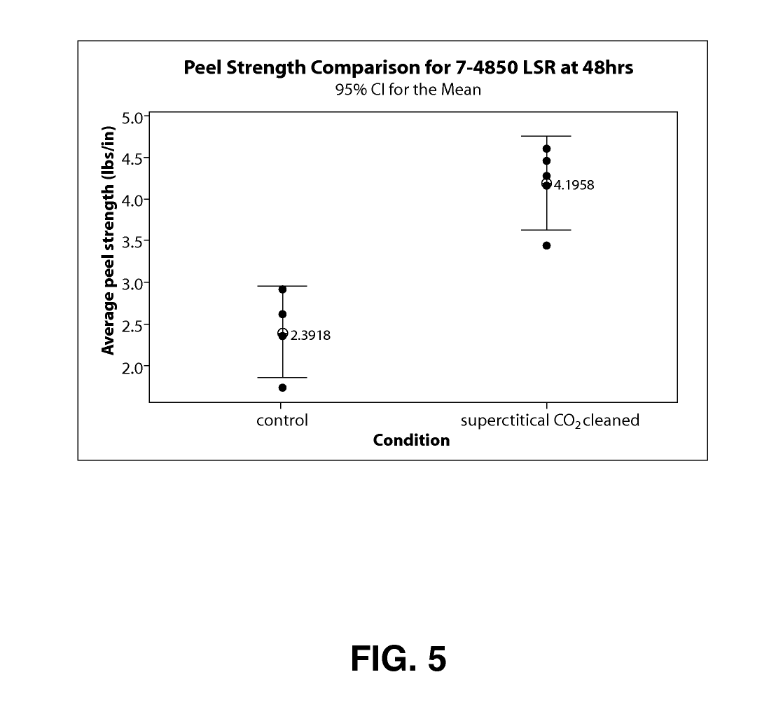

The comparison of blocking in control versus supercritical CO₂ cleaned samples (FIG. 5) at 48 hours show that supercritical CO₂ cleaning increased the rate of blocking. Control samples had mean peel strength of 2.392 pounds (lbs) and the supercritical CO₂ cleaned samples had a mean peel strength of 4.196 lbs. Since the combined sample size for the experiment was ten (10), the effect of Supercritical CO₂ cleaning is proven with 99% confidence.

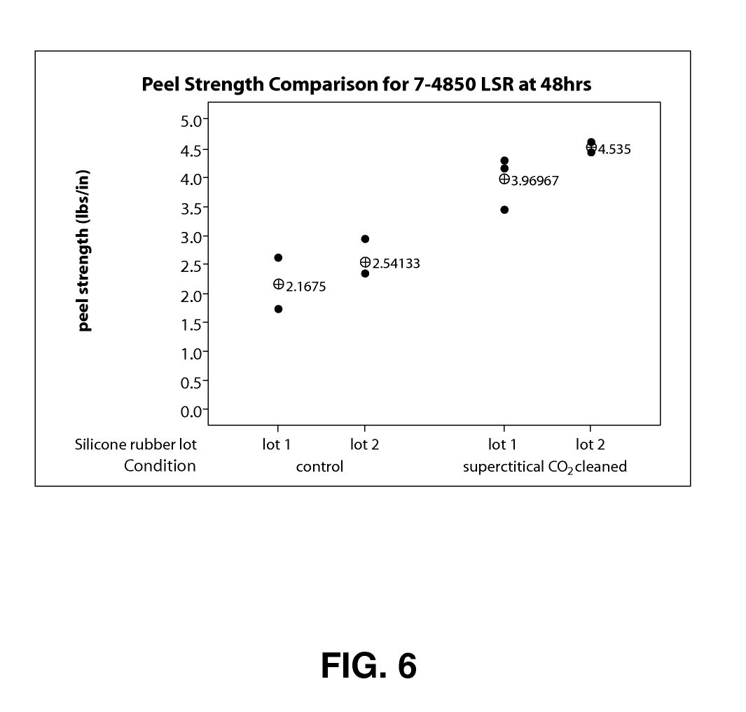

FIG. 6 shows that the increase in blocking rate with supercritical CO₂ cleaning was demonstrated repeatedly with two different lots of silicone rubber. ANOVA (Analysis of Variance) was performed using Minitab 15 on the peel strength data for control and SCCO2 cleaned 50 A LSR at time point 48 hours. The ANOVA results (not shown) indicated that there is a statistically significant difference in peel strength for control and SCCO2 cleaned 50 A LSR, since the p value is less than 0.5.

### Characterization of Control and Supercritical CO₂ Cleaned 7-4850 LSR

Weight loss Data from Extraction. Soxhlet extraction was carried out on the control 7-4850 LSR samples using heptane. Weight loss for the Supercritical CO₂ cleaned 7-4850 LSR was monitored during the Supercritical CO₂ extraction. The weight loss data is given in Table 3.

TABLE 3
Extraction weight loss %

| | Starting weight | Ending weight | % Weight loss |
|---|---|---|---|
| Control (7-4850 LSR) | 14.674 gm | 14.261 gm | 3.65% |
| Supercritical CO₂ | 0.944 gm | 0.910 gm | 3.61% |
| Cleaned (7-4850 LSR) | | | |

From the weight loss data, it can be concluded that supercritical CO₂ cleaning removes the low molecular weight species from LSR.

For the extract from the control 7-4850 LSR, the molecular weight distribution graph obtained from GPC analysis showed three different peaks. A small peak of higher molecular weight was observed. The peak molecular weight of this higher molecular weight distribution was around 105568, based on relative polystyrene distribution. The primary peak in the graph was observed at lower molecular weight, with peak molecular weight at 1049 daltons. There was also a low molecular weight shoulder on the primary peak.

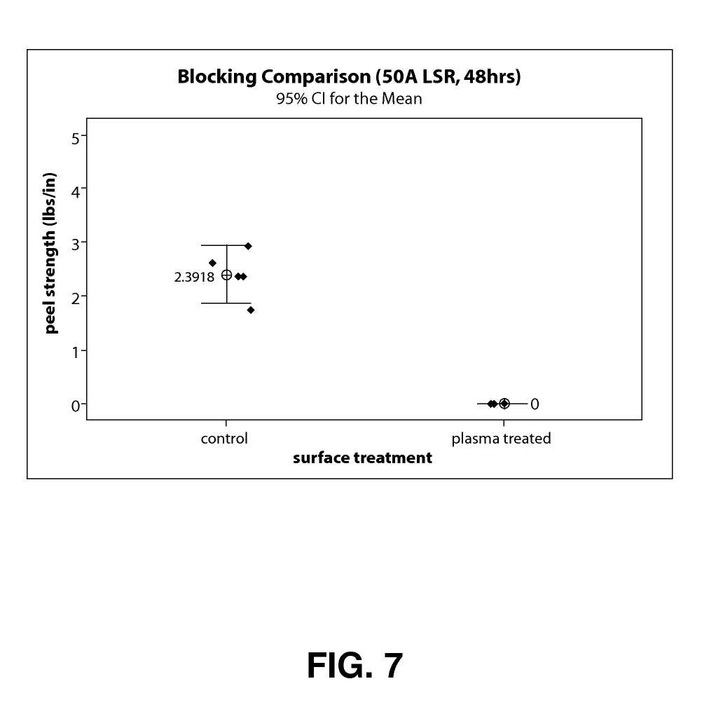

Effect of Plasma Treatment on Blocking. FIG. 7 shows the comparison of the adhesion strength of the plasma-treated 7-4850 LSR and control 7-4850 LSR material at 48 hours. The plasma-treated slabs showed no adhesion at the interface when taken out of the mold. The control 7-4850 LSR slabs had an average adhesion of about 2.392 lbs. It is very evident that Plasma treatment of the silicone rubber material prevented blocking. Since the combined sample size for the experiment was eight (8), the effect of plasma treatment is proven with greater than 95% confidence. ANOVA (Analysis of Variance) was performed using Minitab 15 on the peel strength data for control and plasma-treated 50 A LSR at time point 48 hours. The ANOVA results (not shown) indicated that there is a statistically significant difference in peel strength for control and plasma-treated 50 A LSR, since the p value is less than 0.5.

### Surface Analysis of Plasma-Treated and Control 7-4850 LSR Using Electron Spectroscopy for Chemical Analysis (ESCA)

ESCA analysis was performed to analyze the surface elemental composition on plasma-treated 7-4850 LSR, control 7-4850 LSR material, plasma-treated extracted 7-4850 LSR material and extracted control 7-4850 LSR material. Extraction of the 7-4850 LSR material was done using soxhlet extraction with heptane as the solvent. The purpose of the analysis with extracted samples was to understand the nature of the surface of silicone rubber in the absence of low molecular weight species. High resolution ESCA spectra were obtained on the Si 2p signal to evaluate the binding energy, and ultimately the chemical state of the silicon species. The atomic compositions of the surface (uppermost ˜40 nm) and Si2p binding energy for the samples and times are shown in Tables 4, 5, 6, and 7. The Si2p binding energy of 103.3 eV indicates silica-like (SiO₂) state for silicon and Si2p binding energy of 102.3 eV indicates silicone like (SiO) state for the silicon.

TABLE 4
Relative Atomic % Determined from ESCA Survey
Spectra and Si2p binding energy at time = 0 hrs

| | | | | | | | Si2p |
|---|---|---|---|---|---|---|---|
| | | | | | | | Binding |
| Sample | Area | C | O | F | Si | O/C | (eV) |
| 50A LSR | 1 | 49.4 | 27.3 | 0.3 | 23.0 | 0.55 | 102.3 |
| Control | 2 | 49.2 | 27.3 | 0.2 | 23.3 | 0.55 | 102.3 |
| | 3 | 48.3 | 28.2 | 0.7 | 22.7 | 0.58 | 102.3 |
| | Mean | 49.0 | 27.6 | 0.4 | 23.0 | 0.56 | |
| | Std. Dev. | 0.6 | 0.6 | 0.3 | 0.3 | 0.02 | |
| 50A LSR | 1 | 36.6 | 38.4 | ~ | 24.9 | 1.05 | 102.4 |
| Plasma- | 2 | 41.9 | 33.8 | ~ | 24.3 | 0.81 | 102.3 |
| treated | 3 | 42.9 | 33.0 | ~ | 24.1 | 0.77 | 102.3 |
| | Mean | 40.5 | 35.1 | ~ | 24.5 | 0.87 | |
| | Std. Dev. | 3.4 | 2.9 | ~ | 0.4 | 0.15 | |
| 50A LSR | 1 | 49.7 | 28.7 | ~ | 21.6 | 0.58 | 102.3 |
| Extracted | 2 | 49.3 | 28.7 | ~ | 22.0 | 0.58 | 102.3 |
| Control | 3 | 48.9 | 29.1 | ~ | 22.0 | 0.60 | 102.3 |
| | Mean | 49.3 | 28.8 | ~ | 21.9 | 0.58 | |
| | Std. Dev. | 0.4 | 0.2 | ~ | 0.3 | 0.01 | |
| 50A LSR | 1 | 28.2 | 49.1 | ~ | 22.7 | 1.74 | 103.3 |
| Extracted | 2 | 28.8 | 48.1 | ~ | 23.1 | 1.67 | 103.3 |
| Plasma- | 3 | 26.5 | 50.9 | ~ | 22.6 | 1.92 | 103.3 |
| treated | Mean | 27.9 | 49.4 | ~ | 22.8 | 1.77 | |
| | Std. Dev. | 1.2 | 1.4 | ~ | 0.3 | 0.13 | |

TABLE 5
Relative Atomic % Determined from ESCA Survey
Spectra and Si2p binding energy at time = 4 hrs

| | | | | | | | Si2p |
|---|---|---|---|---|---|---|---|
| | | | | | | | Binding |
| Sample | Area | C | O | F | Si | O/C | (eV) |
| 50A LSR | 1 | 47.8 | 28.3 | ~ | 23.9 | 0.59 | 102.4 |
| Control | 2 | 48.3 | 27.9 | ~ | 23.9 | 0.58 | 102.4 |
| | 3 | 48.0 | 28.2 | ~ | 23.8 | 0.59 | 102.4 |
| | Mean | 48.0 | 28.1 | ~ | 23.9 | 0.59 | |
| | Std. Dev. | 0.2 | 0.2 | ~ | 0.0 | 0.01 | |
| 50A LSR | 1 | 47.5 | 28.8 | ~ | 23.8 | 0.61 | 102.4 |
| Plasma- | 2 | 47.8 | 28.6 | ~ | 23.7 | 0.60 | 102.4 |
| treated | 3 | 48.2 | 28.2 | ~ | 23.6 | 0.58 | 102.4 |
| | Mean | 47.8 | 28.5 | ~ | 23.7 | 0.60 | |
| | Std. Dev. | 0.4 | 0.3 | ~ | 0.1 | 0.01 | |
| 50A LSR | 1 | 51.1 | 27.9 | ~ | 21.0 | 0.55 | 102.4 |
| Extracted | 2 | 50.2 | 28.3 | ~ | 21.5 | 0.56 | 102.4 |
| Control | 3 | 49.9 | 28.7 | ~ | 21.4 | 0.57 | 102.4 |
| | Mean | 50.4 | 28.3 | ~ | 21.3 | 0.56 | |
| | Std. Dev. | 0.6 | 0.4 | ~ | 0.3 | 0.01 | |
| 50A LSR | 1 | 32.0 | 46.5 | ~ | 21.5 | 1.45 | 103.0 |
| Extracted | 2 | 32.5 | 46.2 | ~ | 21.3 | 1.42 | 103.2 |
| Plasma- | 3 | 28.6 | 49.9 | ~ | 21.5 | 1.74 | 103.2 |
| treated | Mean | 31.1 | 47.5 | ~ | 21.4 | 1.53 | |
| | Std. Dev. | 2.1 | 2.1 | ~ | 0.2 | 0.18 | |

TABLE 6
Relative Atomic % Determined from ESCA Survey Spectra
and Si2p binding energy at time = 24 hrs

| | | | | | | | Si2p |
|---|---|---|---|---|---|---|---|
| | | | | | | | Binding |
| Sample | Area | C | O | F | Si | O/C | (eV) |
| 50A LSR | 1 | 49.0 | 27.7 | ~ | 23.3 | 0.57 | 102.4 |
| Control | 2 | 48.5 | 27.9 | ~ | 23.6 | 0.58 | 102.4 |
| | 3 | 48.7 | 27.9 | ~ | 23.4 | 0.57 | 102.4 |
| | Mean | 48.7 | 27.8 | ~ | 23.4 | 0.57 | |
| | Std. Dev. | 0.3 | 0.1 | ~ | 0.2 | 0.01 | |
| 50A LSR | 1 | 48.2 | 28.3 | ~ | 23.6 | 0.59 | 102.4 |
| Plasma- | 2 | 47.7 | 29.0 | ~ | 23.3 | 0.61 | 102.4 |
| treated | 3 | 47.5 | 28.7 | ~ | 23.8 | 0.60 | 102.4 |
| | Mean | 47.8 | 28.6 | ~ | 23.6 | 0.60 | |
| | Std. Dev. | 0.4 | 0.4 | ~ | 0.2 | 0.01 | |
| 50A LSR | 1 | 51.1 | 28.1 | ~ | 20.8 | 0.55 | 102.4 |
| Extracted | 2 | 50.4 | 28.5 | ~ | 21.1 | 0.57 | 102.4 |
| Control | 3 | 50.0 | 28.6 | ~ | 21.4 | 0.57 | 102.4 |
| | Mean | 50.5 | 28.4 | ~ | 21.1 | 0.56 | |
| | Std. Dev. | 0.5 | 0.2 | ~ | 0.3 | 0.01 | |
| 50A LSR | 1 | 32.5 | 44.4 | ~ | 23.1 | 1.37 | 103.2 |
| Extracted | 2 | 33.2 | 44.2 | ~ | 22.6 | 1.33 | 103.2 |
| Plasma- | 3 | 29.0 | 48.6 | ~ | 22.4 | 1.67 | 103.2 |
| treated | Mean | 31.6 | 45.7 | ~ | 22.7 | 1.45 | |
| | Std. Dev. | 2.2 | 2.5 | ~ | 0.4 | 0.19 | |

TABLE 7
Relative Atomic % Determined from ESCA Survey Spectra
and Si2p binding energy at time = 168 hrs

| | | | | | | | Si2p |
|---|---|---|---|---|---|---|---|
| | | | | | | | Binding |
| Sample | Area | C | O | F | Si | O/C | (eV) |
| 50A LSR | 1 | 48.5 | 28.2 | ~ | 23.3 | 0.58 | 102.4 |
| Control | 2 | 48.4 | 28.2 | ~ | 23.4 | 0.58 | 102.4 |
| | 3 | 47.5 | 28.5 | ~ | 24.0 | 0.60 | 102.4 |
| | Mean | 48.1 | 28.3 | ~ | 23.6 | 0.59 | |
| | Std. Dev. | 0.5 | 0.2 | ~ | 0.4 | 0.01 | |
| 50A LSR | 1 | 47.7 | 28.6 | ~ | 23.7 | 0.60 | 102.4 |
| Plasma- | 2 | 47.5 | 28.6 | ~ | 23.9 | 0.60 | 102.4 |
| treated | 3 | 47.4 | 28.6 | ~ | 24.1 | 0.60 | 102.4 |
| | Mean | 47.5 | 28.6 | ~ | 23.9 | 0.60 | |
| | Std. Dev. | 0.2 | 0.0 | ~ | 0.2 | 0.00 | |
| 50A LSR | 1 | 50.5 | 28.7 | ~ | 20.9 | 0.57 | 102.5 |
| Extracted | 2 | 49.1 | 29.5 | ~ | 21.4 | 0.60 | 102.4 |
| Control | 3 | 49.5 | 29.1 | ~ | 21.3 | 0.59 | 102.5 |
| | Mean | 49.7 | 29.1 | ~ | 21.2 | 0.59 | |
| | Std. Dev. | 0.7 | 0.4 | ~ | 0.3 | 0.02 | |
| 50A LSR | 1 | 33.4 | 44.2 | ~ | 22.4 | 1.32 | 103.1 |
| Extracted | 2 | 33.9 | 43.2 | ~ | 22.9 | 1.27 | 103.1 |
| Plasma- | 3 | 30.1 | 47.5 | ~ | 22.3 | 1.58 | 103.1 |
| treated | Mean | 32.5 | 45.0 | ~ | 22.5 | 1.39 | |
| | Std. Dev. | 2.1 | 2.3 | ~ | 0.3 | 0.16 | |

The theoretical composition of 7-4850A LSR by ESCA was 50% C, 25% O, and 25% Si. The control samples showed slightly higher levels of O and lower levels of Si than expected. The plasma-treated samples showed significantly higher O and lower C compared to the controls. To evaluate effect of plasma treatment over time, it is easiest to compare the O/C ratio. The ratio of the atomic concentration of O/C decreased significantly on the two plasma-treated samples during the first four hours (data not shown). The higher O/C ratio is maintained in the extracted sample compared to the non-extracted sample due to absence of migration of low molecular weight species to the surface that covers up the oxygen rich layer.

The Si2p binding energy of approximately 103.3 eV on the 50 A LSR Extracted Plasma-treated sample was higher compared to plasma-treated 7-4850 LSR, control 7-4850 LSR material, and extracted 7-4850 LSR material for which the Si2p binding energy was approximately 102.3 eV. This indicated that the Si on the 50 A LSR Extracted Plasma-treated sample was more silica-like (SiO₂), while the Si on the other samples was more silicone like (SiO). The Si2p binding energy showed only subtle changes with time.

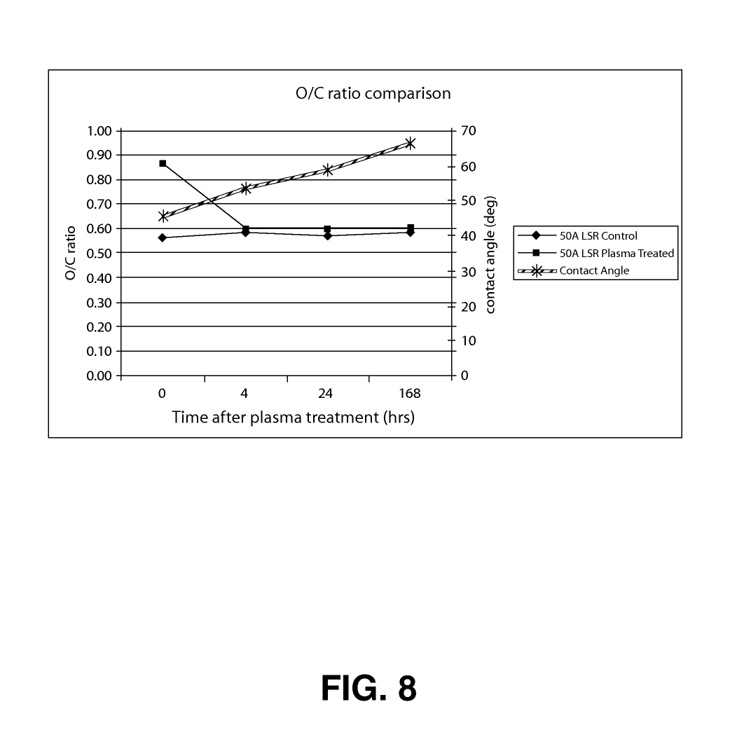

The contact angle versus time for plasma-treated 7-4850 LSR showed that the contact angle increases over time (FIG. 8). Raw data is given in Table 8. Increase in contact angle indicates that hydrophobic recovery occurred. But the plasma-treated sample did not regain the equivalent level of hydrophobicity of the control sample. The control sample had a contact angle of 81.9°. Even at 168 hours (hrs) after plasma treatment, the 7-4850 LSR sample had only a contact angle of 66.5°.

TABLE 8
Contact Angle Measurements

| | | 3.5 hrs | | | |
|---|---|---|---|---|---|
| | 1 hr after | after | 24 hrs after | 168 hrs after | |
| | plasma | plasma | plasma | plasma | |
| | treatment | treatment | treatment | treatment | Control |
| 1 | 44.0 | 53.9 | 55.6 | 69 | 76.6 |
| 2 | 46.9 | 54.5 | 57.8 | 66 | 79.8 |
| 3 | 45.3 | 53.7 | 59.3 | 67.4 | 90.8 |
| 4 | 46.8 | 52.5 | 62.3 | 65.1 | 90.3 |
| 5 | 44.8 | 53.0 | 59.6 | 64.8 | 72.2 |
| AVG | 45.6 | 53.5 | 58.9 | 66.5 | 81.9 |
| Std. Dev. | 1.3 | 0.8 | 2.5 | 1.7 | 8.3 |

The contact angle measurements were consistent with the O/C ratio measurements over time for the plasma-treated samples. As the O/C ratio decreased over time after plasma treatment, the contact angle increased due to hydrophobic recovery. This was due to the surface chemistry created by plasma being buried by low molecular weight mobile silicone species inherent in all silicone formulations. ANOVA (Analysis of Variance) was performed using Minitab 15 on the contact angle data for plasma-treated 50 A LSR at different time intervals after plasma treatment. The ANOVA results (not shown) indicated that there is a statistically significant difference in contact angle for plasma-treated 50 A LSR at different time interval after plasma treatment, since the p value is less than 0.5.

### Blocking Experiment Results Discussion

The primary mechanism for blocking or self adhesion is speculated to be bond interchange at the interface. The bond interchange results in stress relaxation (Stein, J., Stress Relaxation Studies on Unfilled Model Silicone Elastomers, Papers presented at American Chemical Society, Division of Polymer Chemistry, V. 28, issue 2, 1987, p. 377-378; and Gent, A. N., and Vondracek, P., Spontaneous Adhesion of Silicone Rubber, Journal of Applied Polymer Science, 1982, Vol. 27, p. 4357-4364). Factors that affect interfacial bond interchange include molecular mobility (Galliano, A., Bistac, S., and Schultz, J., Adhesion and friction of PDMS networks: molecular weight effects, Journal of Colloid and Interface Science, 265 (2003), p. 372-379) and modulus of the material. Location of low molecular weight species or free chains in the material affects the strength of the adhesion bond.

Increase in molecular mobility favors bond interchange, since the chains are capable of orienting themselves for the interfacial bond interchange. As modulus decreases, bond interchange and consequently blocking increases due to improved substrate wetting. This is due to the chains that participate in bond interchange being in close proximity due to improved wetting. The location of low molecular weight species or the free chains affects the adhesion strength. The presence of low molecular weight species at the interface can reduce blocking since this constitutes a weak boundary layer that reduces the stress transfer from the interface to the network (Galliano, A., Bistac, S., Schultz, J., The role of free chains in adhesion and friction of PDMS networks, The Journal of Adhesion, 2003, 79, p. 973-991).

### Effect of Durometer of Liquid Silicone Rubber on Blocking

Self adhesion or blocking experiments showed that blocking increased with time for 30 A LSR and 50 A LSR. The 7-6830 (the lowest durometer studied) exhibited more blocking than the higher durometers, 7-4870 or 7-4850. The slabs made of 7-4870 material showed the lowest self adhesion strength. The 7-6830 material had lower crosslink density than 7-4870 or 7-4850 material. Also the filler amount was higher in 7-4870. These results indicated that blocking occurs at faster rates in materials that have lower crosslinked density and less amount of filler.

It was noted earlier that blocking occurs due to bond interchange. For bond interchange to occur there has to be sufficient molecular mobility and intimate contact between the chains at the interface. As the crosslink density increases, the chain length between the chemical nodes is reduced. Consequently, chain movement is restricted by the pinning effect of the crosslinks. Also, as the modulus of the material increases substrate wetting is reduced due to reduced proximity/contact of the chains at the interface. This could lead to lower self adhesion strength. Increasing amount of filler causes more obstacles to chain movement. Therefore as filler amount increases, chain movement is reduced and this results in lower rates of blocking. These findings are supported by the results of the blocking experiment with three different durometers (30 A, 50 A, and 70 A) where 30 A had higher self adhesion strength than 50 A and 70 A.

Researchers (Galliano, A., Bistac, S., and Schultz, J., Adhesion and friction of PDMS networks: molecular weight effects, Journal of Colloid and Interface Science, 265 (2003), p. 372-379) have found that adhesion energy has two components: adhesion component and also the dissipative component. When a stress is applied to a polymer, part of the energy is dissipated through chain movements and subsequent energy dissipation. As crosslink density goes up and molecular mobility is restricted by the pinning effect of the crosslinks, energy dissipation in response to a stress goes down at a given testing rate and subsequently the adhesion strength is decreased. In the present work, by adding the mesh in the samples used for the blocking experiment, it was attempted to reduce the contribution of dissipation energy to the adhesion energy. Although the mesh limited the bulk elasticity in the material, there was still minor contribution due to dissipative effects at the interface.

### Effect of Supercritical CO₂ Cleaning on Blocking

In the present work, self adhesion or blocking experiments performed on silicone rubber that was cleaned using supercritical CO₂ showed significantly greater adhesion than the control group of the same durometer (50 A). Supercritical CO₂ cleaning or extraction performed in the present work removed the low molecular weight species from the LSR material (% weight loss=3.61%).

Blocking occurs due to bond interchange at the interface and is influenced by the location of the free chains or low molecular weight species in the material. The low molecular weight species in silicone rubber have lower surface energy than the crosslinked chains. Therefore it is thermodynamically favorable for the low molecular weight free chains to be at the air/polymer interface. It has been reported by researchers (Galliano, A., Bistac, S., Schultz, J., The role of free chains in adhesion and friction of PDMS networks, The Journal of Adhesion, 2003, 79, p. 973-991) that the free chains that are not chemically connected to the network can constitute a weak boundary layer and reduce the adhesion strength by reducing intimate contact between the anchored chains. Also the low molecular weight species at the interface delay the bond exchange across the surface and thereby stress transfer from the interface to the network. This reduces self adhesion strength. The hypothesis in the present work that bond interchange or blocking is affected by location of free chains in the silicone are supported by the results of the blocking experiment where the supercritical CO₂ cleaned samples showed significantly greater adhesion than the control group of the same durometer (50 A).

Researchers (Stein, J., Stress Relaxation Studies on Unfilled Model Silicone Elastomers, Papers presented at Americal Chemical Society, Division of Polymer Chemistry, Vol. 28, Issue 2, 1987, p. 377-378) have concluded that the silicone catalyst plays significant role in self adhesion. However, in the present work the effect of the catalyst (platinum) was separated from the effect of the low molecular weight species on self adhesion. The amount of platinum in the supercritical CO₂ cleaned 7-4850 LSR sample and control 7-4850 LSR sample were compared using ICP analysis. The amount of platinum in both the samples were equivalent (2.0 μg/g in control 7-4850 LSR versus 1.9 μg/g in Supercritical CO₂ cleaned 7-4850 LSR). The degree of self adhesion was drastically different between supercritical CO₂ cleaned 7-4850 LSR sample and control 7-4850 LSR sample. This result indicated that platinum did not play any significant role in the increased adhesion effect in supercritical CO₂ cleaned sample.

### Effect of Plasma Treatment on Blocking

Blocking experiments showed that argon plasma surface treatment of LSR prevented blocking. Researchers (Owen, M. J., and Stasser, J. L., Plasma Treatment of PDMS, American Chemical Society April 1997; and Morra, M., Occhiello, E., Marola, R., Garbassi, F., et al., On the Aging of Oxygen Plasma-Treated Polydimethylsiloxane Surfaces, Journal of Colloid and Interface Science, 1990, Vol. 137, No. 1, p. 11-24) have found that when the control LSR material was plasma-treated oxygen was added to the surface. The oxygen acts to increase the surface energy and this makes it energetically favorable for the low molecular weight species to be at the oxygen enriched silicone/air surface.

As noted earlier, blocking occurs due to bond interchange. One of the factors that affect bond interchange is location of low molecular weight free chains in silicone rubber. The presence of free chains at the interface can constitute a weak boundary layer and reduce the adhesion strength by reducing intimate contact between the anchored chains. Also, the low molecular weight species at the interface delay the bond exchange across the surface and thereby stress transfer from the interface to the network.

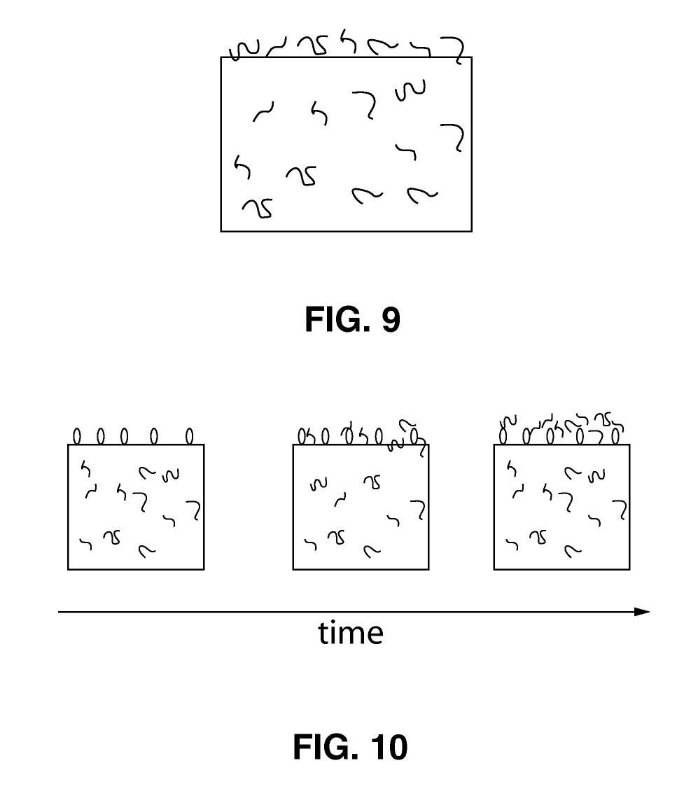

Silicone rubber has approximately 3.65% extractables which consists of low molecular weight species. The low molecular weight species are distributed in the silicone rubber material with more concentration on the surface than the bulk (FIG. 9). The low molecular weight species in silicone rubber have lower surface energy than the crosslinked chains. Therefore, it is thermodynamically favorable for the low molecular weight free chains to be at the air/polymer interface.

Silicone rubber is inherently hydrophobic. With plasma treatment and the creation of an oxygen-rich surface layer, the contact angle at time-0 hr is very low, showing a hydrophilic surface. Over time, the contact angle increases (as evidenced by contact angles and esca) resulting in hydrophobic recovery (FIG. 10). As shown in FIG. 10, on a surface from which the oligomers are cleaned and oxygen is reacted at the surface, hydrophobic recovery is due to migration of low molecular weight species (i.e., oligomers) from the bulk to the surface where they cover the high energy oxygen-rich surface layer. See, for example, Kim, J., Chaudhury, M. K., Owen, M. J., Hydrophobic Recovery of Polydimethylsiloxane Elastomer Exposed to Partial Electrical Discharge, Journal of Colloid and Interface Science, 2000, 226, p. 231-236; Kim, J., Chaudhury, M. K., Owen, M. J., and Orbeck, T., The Mechanisms of Hydrophobic Recovery of Polydimethylsiloxane Elastomers Exposed to Partial Electrical Discharges, Journal of Colloid and Interface Science, 2001, 244, p. 200-207; Morra, M., Occhiello, E., Marola, R., Garbassi, F., et al., On the Aging of Oxygen Plasma-Treated Polydimethylsiloxane Surfaces, Journal of Colloid and Interface Science, 1990, Vol. 137, No. 1, p. 11-24; and Eddington, D. T., Puccinelli, J. P., Beebe, D. J., Thermal aging and reduced hydrophobic recovery of polydimethylsiloxane, Sensors and Actuators, B 114 (2006), p. 170-172. These findings are supported by the results from ESCA and contact angle measurements performed in the present work.

The theoretical composition of control 7-4850 LSR material by ESCA is 50% C: 25% O: 25% Si or a 2:1:1 C:O:Si ratio. The O/C ratio for the control samples (non-extracted and extracted) was around 0.56. The O/C ratio in samples increased up to 0.87 for the non-extracted plasma-treated samples and 1.77 for extracted plasma-treated samples. The amount of silicon however stayed the same. This indicates that plasma treatment changed the surface chemistry of the material. The results show the number of carbon atoms was reduced with corresponding increase in amount of oxygen. This pointed to reduction in carbon atoms bonded to silicon with increase in amount of oxygen bonded to silicon atoms. The O/C ratio, however, reduced over time. The O/C ratio reduced significantly and reached the O/C of the control 7-4850 LSR material in 4 hours. The O/C ratio reduced over time in the plasma-treated extracted 7-4850 LSR, but the O/C ratio did not reach the O/C ratio of the extracted control 7-4850 LSR material. Other researchers have attributed this phenomenon to the low molecular weight species covering the air interface. See, for example, Kim, J., Chaudhury, M. K., Owen, M. J., Hydrophobic Recovery of Polydimethylsiloxane Elastomer Exposed to Partial Electrical Discharge, Journal of Colloid and Interface Science, 2000, 226, p. 231-236; Kim, J., Chaudhury, M. K., Owen, M. J., and Orbeck, T., The Mechanisms of Hydrophobic Recovery of Polydimethylsiloxane Elastomers Exposed to Partial Electrical Discharges, Journal of Colloid and Interface Science, 2001, 244, p. 200-207; Morra, M., Occhiello, E., Marola, R., Garbassi, F., et al., On the Aging of Oxygen Plasma-Treated Polydimethylsiloxane Surfaces, Journal of Colloid and Interface Science, 1990, Vol. 137, No. 1, p. 11-24. The plasma treatment increased the O/C ratio. But over time, in the control 7-4850 LSR material, the low molecular weight species migrated to the surface to cover the high-energy oxygen-rich surface. This was also confirmed with the contact angle measurements.

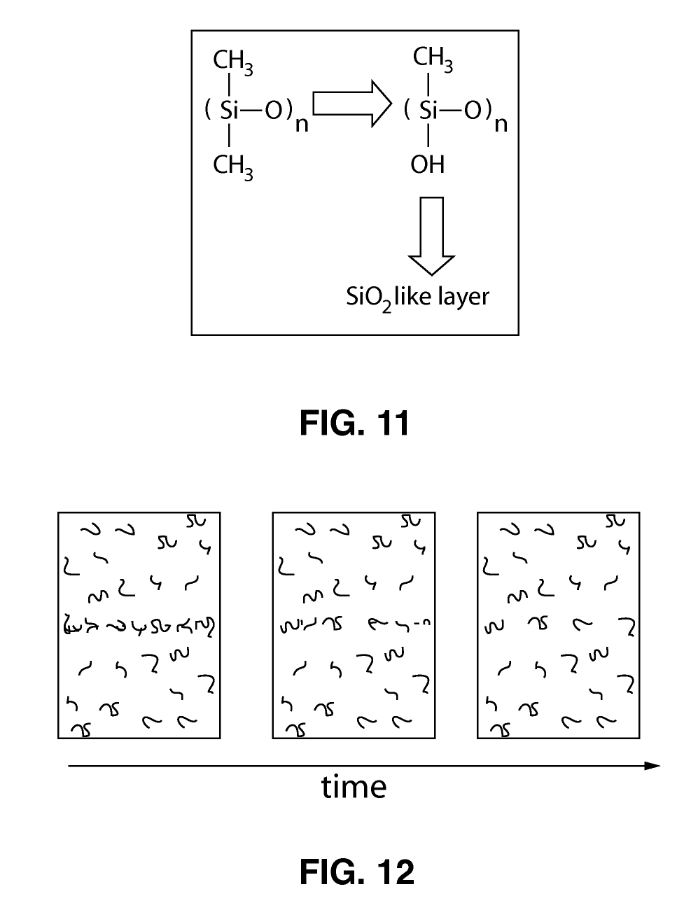

The proposed mechanism for creation of the oxygen rich layer on the plasma-treated silicone surface is illustrated in FIG. 11. During plasma treatment, radicals were formed in the polymer chain. The free radicals reacted with oxygen in the chamber and resulted in creation of oxygen functionalities. The proposed mechanism is oxidation of SiMe to SiOH (silanols). The silanols condensed to form the silica-like layer on the surface.

In the present work, ESCA analysis show that the Si2p binding energy shifted in the plasma-treated extracted 7-4850 LSR sample to approximately 103.2 eV compared to 102.4 eV for the control samples. This indicated that the silicon is more silica-like in the plasma-treated extracted 7-4850 LSR sample compared to silicone-like in the other three samples. These results confirm the hypothesis that the oxygen on the plasma-treated surface is silica-like.

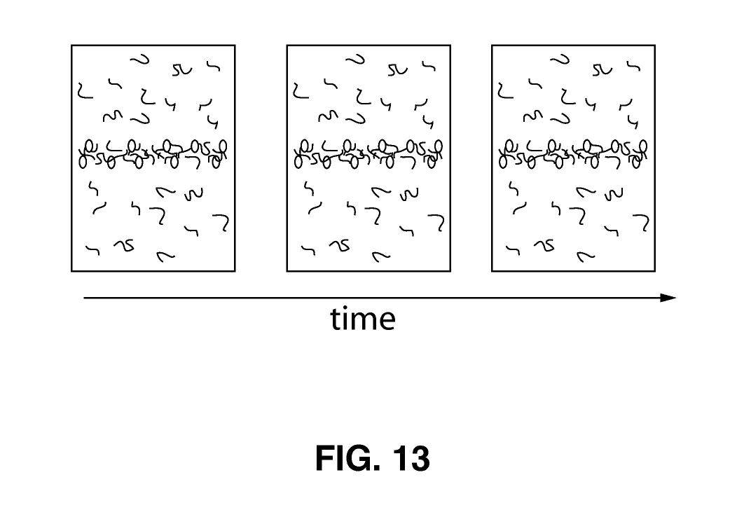

The proposed mechanism for the elimination of blocking by argon plasma treatment is as follows. Upon mating the control 7-4850 LSR control sample, the interface is initially enriched with low molecular weight species that was driven to the surface due to its air interface. Over time during the blocking experiment, the low molecular weight species redistributed uniformly into the bulk (10), due to the lack of favorable thermodynamics which is a result of the absence of air at the interface. Self healing occurred between anchored (crosslinked) chains (FIG. 12). The plasma-treated samples also have an interface enriched with low molecular weight species that was driven to the surface due to its high surface energetics of the oxidized silica-like surface. However, unlike the control material, the permanent silica-like layer keeps the interface defined even in the absence of air. That is, when the two plasma-treated surfaces are mated, the energetically favorable position for the low molecular weight species is still on the surface of the silica-like layer at the interface. The consequence was a weak boundary layer that prevented blocking (FIG. 13) in the plasma-treated samples. This proposed mechanism is supported by the results of the blocking experiments with control and plasma-treated LSR material in the present work where it was shown that blocking is prevented by plasma treatment of silicone rubber.

### Grommet Punchout Test

Grommet punchout is caused by blocking (e.g., self-adhesion of silicone). A high level of blocking causes a wrench to punch out a plug of material (e.g., silicone) when the bottom of the grommet contacts the set screw. This is a punch and die effect and is undesirable.

A grommet can be tested by isolating the grommet without set screws and inserting a wrench using an INSTRON to measure the force of insertion. This can be compared against a “freshly made” grommet that was not plasma treated, which does not demonstrate blocking. The average energy of grommets tested in devices that punched out was 0.056 in-lbs, whereas for grommets that did not punch out, the average energy was 0.047 in-lbs. Preferably, a grommet plasma-treated according to the present invention performs similar to an untreated “freshly made” grommet.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A medical device comprising a sealing apparatus that comprises a first element and a second element, wherein the first element comprises a first surface and the second element comprises a second surface, wherein the first surface of the first element faces and is in physical contact with the second surface of the second element, and wherein the first element comprises an organic polymer and has a bulk durometer value on the Shore A scale, and further wherein the first surface is a plasma-treated surface.

2. The medical device of claim 1, wherein the first element and the second element comprise portions of a single, integral body.

3. The medical device of claim 1, wherein each of the first and second surfaces is a plasma-treated surface.

4. The medical device of claim 1, wherein the first element comprises a mobile species that is immobilized at the plasma-treated surface.

5. The medical device of claim 1, which is implantable.

6. The medical device of claim 5, wherein the implantable medical device is selected from stimulators, pacemakers, defibrillators, drug pumps, and implantable pulse generators.

7. The medical device of claim 6, wherein the sealing apparatus comprises a grommet for securing a lead to the device.

8. The medical device of claim 1, wherein the sealing apparatus comprises a plunger in a syringe.

9. The medical device of claim 1, wherein the sealing apparatus comprises a valve and the first and second surfaces form a valve slit.

10. The medical device of claim 1, wherein the first element comprises silicone, ethylene propylene diene monomer, butyl rubber, fluorine elastomers, and combinations thereof.

11. The medical device of claim 1, wherein the second element comprises a metal, glass, or an organic polymer.

12. The medical device of claim 11, wherein the second element comprises an organic polymer and has a bulk durometer value on the Shore A scale.

13. The medical device of claim 12, wherein the second element comprises silicone, ethylene propylene diene monomer, butyl rubber, fluorine elastomers, and combinations thereof.

14. The medical device of claim 12, wherein the first element comprises an organic polymer having a bulk durometer value of at least 30 A, and the second element comprises an organic polymer having a bulk durometer value of at least 30 A.

15. The medical device of claim 1, wherein the first and second elements comprise silicone and the first and second surfaces form an interface under a compressive stress.

16. The medical device of claim 15, wherein the silicone elements comprise oligomers having a molecular weight less than the entanglement molecular weight of the silicone, and the interface comprises a region of a higher concentration of the oligomers relative to the remainder of the silicone elements.

17. The medical device of claim 1, wherein the first and second surfaces form an interface and display a lower peel strength 48 hours after formation of the interface than a control, wherein the control includes an interface formed of the same first and second elements without the plasma-treated surface.

18. A medical device comprising a sealing element, wherein the sealing element comprises two surfaces forming an interface under a compressive stress, wherein at least one surface at the interface comprises an organic polymeric material having a bulk durometer value on the Shore A scale and is plasma-treated.

19. The medical device of claim 18, wherein the sealing element comprises a mobile species that is immobilized at the interface.

20. The medical device of claim 18, wherein the two surfaces forming an interface are polymeric surfaces.

21. The medical device of claim 20, wherein the polymeric surfaces comprise silicone.

22. The medical device of claim 18, which is implantable.

23. The medical device of claim 22, wherein the implantable medical device is selected from stimulators, pacemakers, defibrillators, drug pumps, and implantable pulse generators.

24. The medical device of claim 23, wherein the sealing apparatus comprises a grommet for securing a lead to the device.

25. The medical device of claim 18, wherein the sealing element comprises two surfaces having a bulk durometer value of at least 30 A.

26. A method of making a medical device comprising a sealing apparatus, the method comprising:
providing a first element comprising an organic polymer having a bulk durometer value on the A scale, wherein the first element has a first surface;
providing a second element, wherein the second element has a second surface;
treating the first surface of the first organic polymeric element with a plasma to form a plasma-treated surface; and
contacting the plasma-treated surface of the first polymeric element with the second surface of the second element to form a sealing apparatus.

27. The method of claim 26, wherein treating the first surface with a plasma creates an oxygen-rich surface.

28. The method of claim 26, wherein the plasma is a radio frequency induced plasma.

29. The method of claim 28, wherein the plasma is generated at a frequency of about 13.56 MHz and at 150 mTorr.

30. The method of claim 26, wherein the plasma treatment takes place in the presence of a gas selected from the group consisting of hydrogen, nitrogen, helium, argon, neon and mixtures thereof.

31. The method of claim 26, wherein treating the first surface of the first polymeric element with a plasma to form a plasma-treated surface is carried out for a time sufficient to prevent blocking.

32. The method of claim 26, wherein the first and second surfaces form an interface and display a lower peel strength 48 hours after formation of the interface than a control, wherein the control includes an interface formed of the same first and second elements without the plasma-treated surface.

33. The method of claim 26, wherein the second element comprises an organic polymer having a bulk durometer value on the A scale.

34. The method of claim 33, wherein the first element comprises an organic polymer and has a bulk durometer value of at least 30 A, and wherein the second element comprises an organic polymer and has a bulk durometer value of at least 30 A.
